(19) United States
(12) Patent Application Publication
(10) Pub. No.: US 2011/0195120 A2
(43) Pub. Date: Aug. 11, 2011

(54) Sustained Release Pharmaceutical Composition Containing Metformin Hydrochloride

(75) Inventors: Suresh GIDWANI, Mumbai (IN); Purushottam Singnurkar, MUMBAI (IN); Prashant Tewari, Mumbai (IN)

(73) Assignee: USV Ltd., Mumbai (IN)

(21) Appl. No.: 10/272,812

(22) Filed: Oct. 17, 2002

Related U.S. Application Data
(63) Continuation of application No. 09/857,077, filed on Apr. 2, 2002.

(51) Int. Cl. A61K 9/22

(52) U.S. Cl. 424/468

(57) ABSTRACT

A monolithic sustained-release pharmaceutical composition comprising a therapeutically effective dose of metformin hydrochloride as an active substance and a hydrophobic polymer and/or other hydrophobic material, wherein the metformin hydrochloride is released no more than forty percent in gastric fluid having pH 1.2 and is released no less than ninety percent eight to ten hours after administration in simulated intestinal fluid (phosphate buffer) having pH 6.8, and wherein the metformin hydrochloride displays a peak plasma concentration, a systemic bioavailability over time, and a residual plasma concentration twenty-four hours after administration of an oral dosage form of the pharmaceutical composition, so that the metformin hydrochloride concentration remains therapeutically effective and once daily administration of the pharmaceutical composition is sufficient to be therapeutically effective throughout a day.

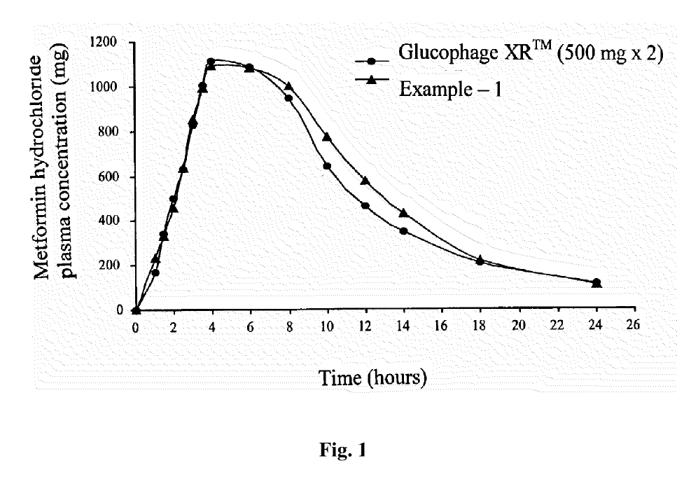

### CROSS-REFERENCE TO RELATED APPLICATIONS

Ser. No. 09/857,077, filed Apr. 2, 2002, for SUSTAINED RELEASE PHARMACEUTICAL COMPOSITIONS CONTAINING METFORMIN AND METHOD OF THEIR PRODUCTION, now pending, which is a national phase entry under 35 U.S.C. §371 of PCT/IB00/01404 filed Oct. 2, 2000, now pending.

### BACKGROUND OF THE INVENTION

1. Field of the Invention

The present invention relates to a sustained-release pharmaceutical preparation containing metformin hydrochloride, which provides a sustained release of metformin hydrochloride over a prolonged period of time, and a method of producing same.

2. Discussion of the Related Art

Metformin hydrochloride is known as a biguanide derivative (1,1-dimethylbiguanide monohydrochloride), and is widely used as an oral antihyperglycemic agent in the management of non-insulin-dependent diabetes mellitus (NIDDM). Metformin hydrochloride is highly water soluble (>300 mg/ml at 25° C.), which contributes to the difficulty in making a sustained release dosage form thereof.

Commercially available preparations of metformin hydrochloride, such as those having a dosage of 850 mg and labeled “retard tablets” (i.e., Glucophage® RTM retard), have not proven to be advantageous in limited volunteer trials. This is likely due to a poor choice of polymers and a lower dosage than that desired for sustained action.

It is known in the art to produce an 850 mg metformin hydrochloride retard tablet containing hydrocolloid-forming retarding agents, with further control of metformin release provided by a film envelope. However, no justification is provided for the 850 mg dose as it relates to a delayed-release preparation of metformin and the expected release rates from such compositions. Given that the relevant literature indicates that metformin hydrochloride has only forty to sixty percent (40%-60%) bioavailability with high renal clearance, an 850 mg dose may be insufficient to achieve the therapeutic plasma concentration of approximately 1 μg/ml for a sufficient period of time. Thus, such a dosage may require twice or even thrice daily administration.

Also known in the art is a biphasic controlled-release delivery system for metformin hydrochloride, comprising a tablet with an inner solid particulate phase and an outer solid continuous phase utilizing hydrophilic and hydrophobic polymers. The tablet is hydrodynamically balanced, and swells to approximately three times its dry size following hydration. However, it is known that, where the subject is in a supine position, the tablet escapes through the pylorus of the stomach after administration. This may diminish the tablet's in vivo performance. Additionally, the volume of stomach contents required to maintain the tablet as floating is sufficient only in the fed condition. An additional limitation relates to the dosage of the metformin hydrochloride and its formulation, with each 1.0 g tablet containing only approximately 500 mg metformin hydrochloride. Administration of two tablets each time is thus required to provide the desired sustained action.

What is needed is a sustained release pharmaceutical composition containing metformin hydrochloride as an active ingredient, wherein the high water solubility of the metformin hydrochloride is limited, and wherein the metformin is released no more than 40% in the gastric fluid and no less than 90% in the intestinal fluid. What is further needed is a sustained release pharmaceutical composition containing metformin hydrochloride as an active ingredient, wherein the metformin hydrochloride displays a peak plasma concentration and systemic bioavailability sufficient to permit once daily administration. What is further needed is a sustained release pharmaceutical composition containing metformin hydrochloride as an active ingredient, wherein the metformin hydrochloride displays a peak plasma concentration of at least 1 μg/ml and a systemic bioavailability such that the residual metformin plasma concentration 24 hours after administration of an oral dosage form of the composition containing 1000 mg of metformin hydrochloride is at least 100 ng/ml.

Accordingly, the present invention provides a sustained release pharmaceutical composition containing metformin hydrochloride, wherein the high water solubility of the metformin hydrochloride is limited, and wherein the metformin is released no more than 40% in the gastric fluid and no less than 90% in the intestinal fluid. The present invention also provides a sustained release pharmaceutical composition containing metformin hydrochloride as an active ingredient, wherein the metformin hydrochloride displays a peak plasma concentration and systemic bioavailability sufficient to permit once daily administration. The present invention also provides a sustained release pharmaceutical composition containing metformin hydrochloride as an active ingredient, wherein the metformin hydrochloride displays a peak plasma concentration of at least 1 μg/ml and a systemic bioavailability such that the residual metformin plasma concentration 24 hours after administration of an oral dosage form of the composition containing 1000 mg of metformin hydrochloride is at least 100 ng/ml.

The present invention is based on calculation of the dose of metformin hydrochloride desired, as determined by in vivo studies documented in the literature. The model here is based on the equations of Dobrinska and Welling (1975), which provides accurate calculations of loading dose and maintenance dose for achieving the desired sustained release effect. The invention is confirmed by subsequent in vivo bioavailability and bioequivalence studies, showing that a single dose of the pharmaceutical composition of the present invention (a) has a sustained systemic bioavailability similar to that of an immediate release metformin hydrochloride tablet, and (b) provides as much metformin hydrochloride as two 500 mg tablets, with respect to peak plasma concentration and systemic bioavailability, thus rendering the composition of the present invention suitable for once daily administration.

The dose of metformin hydrochloride is calculated based on the following pharmacokinetic values, taken from the literature:

| | | |
|---|---|---|
| Peak plasma concentration (Cₘₐₓ): | 1.02 | μg/ml |
| Elimination half-life (t½) | 6.2 | hours |
| Volume of distribution (V_{d}) | 275 | ml |
| Renal clearance: | 552 ± 139 | ml/min |
| Total clearance: | 1300 | ml/min |

Using the Dobrinska and Welling model, the calculated loading dose of metformin hydrochloride is 283 mg, the maintenance dose is 759 mg, and the total dose of metformin hydrochloride is 1040 mg, for achieving the sustained release effect for 24 hours. The aforementioned bioavailability and bioequivalence studies confirm that a total dose of 1000 mg of metformin, formulated according to the method described herein, is sufficient to provide a therapeutic dose of metformin hydrochloride over a period of 24 hours, based on a single oral administration.

### BRIEF SUMMARY OF THE INVENTION

The sustained release pharmaceutical composition of the present invention comprises metformin hydrochloride as the active substance in combination with a hydrophobic polymer and/or other hydrophobic material. The composition is formulated so that the metformin hydrochloride is released no more than forty percent (40%) in gastric fluid, pH 1.2, and no less than ninety percent (90%) eight to ten hours after administration in a simulated intestinal fluid, pH 6.8. The metformin hydrochloride displays a peak plasma concentration (Cₘₐₓ), a systemic bioavailability over time (AUC₀₋₂₄), and a residual plasma concentration twenty-four hours after administration of an oral dosage form of the composition, so that the metformin hydrochloride concentration remains therapeutically effective and once daily administration of the pharmaceutical composition is sufficient to be therapeutically effective for the entire time.

In a preferred embodiment, the sustained release pharmaceutical composition of the present invention is formulated such that the metformin hydrochloride displays peak plasma concentration of at least 1 μg/ml and has a systemic bioavailability such that the residual metformin plasma concentration 24 hours after administration of an oral dosage form of the composition containing 1000 mg of metformin hydrochloride is at least 100 ng/ml.

### BRIEF DESCRIPTION OF THE DRAWING

The drawing is a graph showing metformin hydrochloride plasma concentration as a function of time, comparing a single dose of the metformin hydrochloride-containing pharmaceutical composition of the present invention with two (2) doses of a commercially available metformin composition, Glucophage XR™.

### DETAILED DESCRIPTION OF THE INVENTION

The present invention provides a monolithic sustained-release pharmaceutical composition comprising a therapeutically effective dose of metformin hydrochloride as an active substance and a hydrophobic polymer and/or other hydrophobic material, wherein the metformin hydrochloride is released no more than forty percent in a gastric fluid having pH 1.2 and is released no less than ninety percent eight to ten hours after administration in a simulated intestinal fluid having pH 6.8. The metformin hydrochloride displays a peak plasma concentration, a systemic bioavailability over time, and a residual metformin plasma concentration twenty-four (24) hours after administration of an oral dosage form of the pharmaceutical composition, so that the metformin hydrochloride concentration remains therapeutically effective and once daily administration of the pharmaceutical composition is sufficient to be therapeutically effective for the entire time period.

The pharmaceutical composition of the present invention is formulated to be palatable and swallowable, and provides a simple monolithic system composed of approximately 1000 mg of metformin hydrochloride in combination with hydrophobic polymers and other excipients with improved kinetics of extended-release dosage forms, and with the highest possible content of active ingredient and the simplest method of production.

The composition comprises approximately 60 to 90 percent (by weight) of active substance, and preferably 70 to 80 percent (by weight) of the active substance, and one or more hydrophobic polymer and/or other hydrophobic material in an amount comprising approximately 10 to 40 percent (by weight), and preferably 20 to 30 percent (by weight), based on the weight of the active substance.

Hydrophobic polymers which may be employed for the pharmaceutical composition include, but are not limited to, stearic acid, glyceryl monostearate, glyceryl behenate, glyceryl monooleate, glyceryl palmitostearate, microcrystalline wax, stearyl alcohol, cetyl alcohol, cetostearyl alcohol, hydrogenated castor oil, tristearin, waxes, polyethylene powder, polyvinyl chloride, shellac, rosin, and similar substances. Generally, the hydrophilic polymers and/or other substances may be selected from fatty acids, fatty alcohols, fatty acid esters, hydrogenated oils, waxes, and natural resins. When the hydrophobic polymer will be employed as a mixture with other hydrophobic materials, the weight range of hydrophobic polymer to other hydrophobic material is from 1:0.1 to 1:5, and preferably about 1:0.3.

The pharmaceutical composition of the present invention may be used to produce any of the conventional oral dosage forms, including, without exception, tablets of any shape, preferably oval. The composition additionally may be coated with a film coat of commonly used hydrophilic-containing polymers. The film envelope used can be a taste-neutral film-forming agent, to which dyes can optionally be added for increased aesthetic enjoyment. The proportion by weight of the film envelope relative to the final tablet weight is approximately 0.5 to approximately 4 percent by weight, and preferably about 1.0 to about 1.5 percent by weight. The film may be formed from conventional film-forming substances, such as hydroxypropyl methylcellulose, hydroxypropyl cellulose, starch, cellulose derivatives, and the like.

The pharmaceutical composition of the present invention can also be used to produce compressed slugs and filled into capsules.

Auxiliary substances which may be employed for the pharmaceutical composition include binders, glidants and lubricants. The binders may include, but are not limited to, polyvinyl pyrollidone, gelatin, gum acacia, Klucel® EF (hydroxypropyl cellulose), carboxymethyl cellulose sodium, and any combination thereof. Glidants may include, but are not limited to, colloidal silicone dioxide, talc, starch and any combination thereof. Lubricants include, but are not limited to, magnesium stearate, zinc stearate and any combination thereof.

Apart from the active substance and hydrophobic polymers and/or other hydrophobic substances, an oral dosage form of the pharmaceutical composition of the present invention may contain 1.0 to 15 percent by weight of binder, preferably 3.0 to 10 percent by weight; up to 2.0 percent by weight of glidant, and preferably 0.5 to 1.0 percent by weight; and up to 2.0 percent by weight of lubricant, and preferably 0.5 to 1.0 percent by weight, each of the foregoing in relation to the overall weight of the oral dosage form.

The pharmaceutical composition of the present invention, in the oral dosage form such as a tablet, is produced by dry mixing the active substance, metformin hydrochloride, and optionally further auxiliary substances, and granulating this mixture with hydrophobic polymers and/or other hydrophobic materials by hot melt granulation technique using a jacketed rapid mixer granulator at a temperature of 40 to 120° C., preferably 60 to 80° C. The granulated mixture is cooled to room temperature with continuous mixing. The resulting mass is further granulated with an aqueous or organic solution of the binder, followed by drying and converting to 30 μm to 2.0 mm granules, preferably 100 μm to 1.0 mm, by milling and sizing. Subsequently, appropriate other pharmaceutical auxiliary substances are admixed to the sized granules.

Alternatively, the active substance may be dry mixed with further auxiliary substances, hydrophobic polymers and/or other hydrophobic materials, and a binder, in an extruder. The resulting mix is extruded at a temperature of 40 to 120° C., and preferably 60 to 90° C., in a simple extruder, such as those used for injection molding of plastics. The extruded molten mass is cooled to room temperature and converted to 30 μm to 2.0 mm granules, preferably 100 μm to 1.0 mm, by milling and sizing. Subsequently, appropriate other pharmaceutical auxiliary substances may be admixed with the sized granules.

The pharmaceutical composition of the present invention, produced in this manner, is subsequently processed by conventional methods to produce the oral dosage forms, i.e., compressing into tablets, or filling pressed slugs into capsules. Tablets can be coated with a film using conventional coating processes and methods, such as conventional pan or fluid coating.

The sustained released pharmaceutical composition of the present invention releases metformin hydrochloride in a controlled manner, thereby providing a therapeutic effect over a time period of up to 24 hours, and preferably over a time period of 18 hours.

Useful pharmaceutical compositions according to the present invention demonstrate the following in vitro drug release characteristics when tested in gastric fluid having pH of 1.2 for the first hour, and then in phosphate buffer having pH of 6.8 (simulating intestinal fluid):

| Time (hours) | Percent Release |
|---|---|
| 1 | 38-45 |
| 2 | 50-55 |
| 3 | 62-68 |
| 4 | 70-75 |
| 5 | 80-85 |
| 6 | 85-90 |
| 7 | 91-95 |
| 8 | 96-100 |

In a preferred embodiment, the pharmaceutical composition of the present invention was formed as follows: 225 g of stearic acid was melted at 70° C. 1000 g of metformin hydrochloride was heated to 70° C. in a jacketed rapid mixer granulator and granulated with the melted stearic acid at 70° C. After granulation, the granulated mass was mixed continuously with gradual cooling to room temperature.

60 g of shellac and 25 g of polyvinyl pyrollidone were dissolved in 150 g of isopropyl alcohol. The solution was gradually added to the metformin-stearic acid granulate and mixed until a dough mass formed. The dough mass was dried at 45° C. for 2 hours, and then sized through 2.4 mm screen to break the agglomerates. The sized granules (1310 g) were blended with 4.0 g of colloidal silicone dioxide and 8.0 g magnesium stearate, and compressed into capsule-shaped oval tablets, each containing 1000 mg of metformin hydrochloride.

The in vitro release characteristics of the above tablets were as follows:

| Time (Hrs) | Percent Release |
|---|---|
| 1 | 40 |
| 2 | 55 |
| 3 | 65 |
| 4 | 75 |
| 5 | 82 |
| 6 | 89 |
| 7 | 95 |
| 8 | 99.5 |

The tablets produced as described were then subjected to both bioavailability and bioequivalence studies.

The bioavailability study consisted of a randomized, two-treatment, two-way, two-period, cross-over comparative bioavailability of a single dose of the above produced pharmaceutical composition (Metformin SR 1000 tablets) against a conventional immediate release tablet containing 1000 mg of metformin hydrochloride. Testing was conducted on six healthy adult male human subjects under fasting conditions. Blood samples were taken at 0, 1, 1.5, 2, 2.5, 3, 3.5, 4, 6, 8, 10, 12, 14, 18 and 24 hours, and analyzed for the presence of metformin. The mean plasma profile demonstrated a useful modification of drug release in vivo relative to the immediate-release formulation, with no impact on bioavailability: the AUC₀₋₂₄ for the sustained release pharmaceutical composition prepared as above was 86.22 percent of that of the AUC₀₋₂₄ obtained with the conventional, immediate release tablet. This was found to be well within the acceptable range of 80 to 120 percent. The following table shows the comparable Cₘₐₓ and AUC₀₋₂₄ values for the immediate-release tablet and the sustained-release tablet of the present invention (Metformin SR 1000):

| | Cₘₐₓ | AUC₀₋₂₄ |
|---|---|---|
| Formulation | ng/ml | ng/ml/hr |
| Metformin tablet, 1000 mg, | 1837 ± 33.8 | 13473 ± 19.2 |
| immediate release | | |
| Metformin SR 1000 | 1281 ± 22.1 | 11794 ± 29.6 |

The bioequivalence study consisted of a randomized, two-treatment, two-way, two-period, cross-over comparative bioavailability of a single dose of the above produced pharmaceutical composition (Metformin SR 1000 tablets) against two tablets of a commercially available metformin-containing tablet (Glucophage XR™ 500 mg tablets, manufactured by Bristol Myers-Squibb). Testing was conducted on 12 healthy adult male human subjects under fed conditions. Comparative pharmacokinetic parameters are listed in the following table:

| | Cₘₐₓ | AUC₀₋₂₄ | AUC₀₋₀₀ | |
|---|---|---|---|---|
| Formulation | ng/ml | ng/ml/hr | ng/ml/hr | Tₘₐₓ |
| Metformin SR 1000 | 1302 ± 24.63 | 12653 ± 36.74 | 13387 ± 36.55 | 5.83 ± 0.63 |
| Glucophage XRTM 500 | 1265 ± 25.6 | 11844 ± 35.19 | 12739 ± 36.64 | 4.92 ± 0.48 |
| mg (double dose) | | | | |

As shown, the Metformin SR tablets, comprised of the pharmaceutical composition of the present invention produced as per above, were found to be bioequivalent to two 500 mg tablets of Glucophage XR™ 500 with respect to Cₘₐₓ and AUC.

The pharmacokinetic data based on bioavailability and bioequivalence thus suggest that the pharmaceutical composition of the present invention (in the form of Metformin SR 1000 mg tablet) is suitable for once daily administration, as further indicated by the sufficient residual plasma concentration level of metformin after 24 hours, viz., approximately 100-200 ng/ml. See the drawing.

Indeed, as shown in the drawing, a single oral dosage form of the pharmaceutical composition of the present invention containing 1000 mg of metformin hydrochloride, displays a bioavailability over time and a metformin plasma concentration over time nearly identical to the corresponding parameters for two 500 mg dosage forms of Glucophage XR™. That is, the plasma concentration of metformin increases over the first four hours after administration to a peak plasma concentration (Cₘₐₓ) of approximately 1100 ng/ml, and then decreases gradually, in a generally linear fashion, such that the residual plasma concentration of metformin 24 hours after administration is between 100 ng/ml and 200 ng/ml, or between about 8 and 18 percent (8-18%) of the Cₘₐₓ, and more precisely is approximately 115 ng/ml, or approximately ten percent (10%) of the Cₘₐₓ. A single oral dosage form of the pharmaceutical composition containing 1000 mg of metformin hydrochloride thus possesses sufficient sustained release pharmacokinetics to allow for once daily administration to be therapeutically effective.

While the invention has been described with respect to certain specific embodiments, it will be appreciated that many modifications and changes may be made by those skilled in the art without departing from the invention. It is intended, therefore, by the appended claims, to cover all such modifications and changes as may fall within the true spirit and scope of the invention.

We claim:

1.-8. (canceled)

9. A monolithic sustained release pharmaceutical composition as tablets for once daily use, consisting essentially of 1000 mg metformin hydrochloride as the active substance, 10 to 40% by weight of a hydrophobic polymer and/or other hydrophobic material, 3 to 10% by weight of a binder, 0.5 to 1.5% by weight of a glidant and 0.5 to 1.0% by weight of a lubricant, said tablets having a film coating for taste neutralization, the in-vitro drug release characteristics of said tablets when tested in gastric fluid of pH 1.2 for the first hour and then in phosphate buffer of pH 6.8 USP for the remaining 7 hours being as follows:

| time in | % of drug |
|---|---|
| hours | release |
| 1 | 38-45% |
| 2 | 50-55% |
| 3 | 62-68% |
| 4 | 70-75% |
| 5 | 80-85% |
| 6 | 85-90% |
| 7 | 91-95% |
| 8 | 96-100% |

10.-15. (canceled)

16. A method of treatment in the management of noninsulin dependent diabetes mellitus (NIDDM) comprising administering to a patient in need of such treatment a monolithic sustained release pharmaceutical composition of claim 9 in the form of a tablet containing 1000 mg. of metformin hydrochloride, said tablet having a film coating for taste neutralization.

17.-20. (canceled)

21. A pharmaceutical tablet comprising:
a. At least about one gram of metformin hydrochloride,
b. Hydrophobic material in an amount of up to about 40% (w/w) of the amount of said metformin hydrochloride,
c. Hydrophilic material in an amount of not more than about 5% (w/w) of said metformin hydrochloride,
d. Said pharmaceutical tablet providing extended release.

22. The tablet of claim 21, having a variability of drug release rate of not more than about ±3.5% during any one hour.

23. The tablet of claim 21, having an average variability of drug release for the maintenance dose of not more than about ±2.4%.

24. The tablet of claim 21, having an average variability of drug release for the loading dose and the maintenance dose of not more than about ±2.6%.

25. The pharmaceutical tablet of claim 24, wherein the variability of drug release for the loading dose is not more than about ±3.5%.

26. The tablet of claim 25 having approximately the following variability in drug release:

| time in hours | variability of drug release |
|---|---|
| 1 | ±3.5% |
| 2 | ±2.5% |
| 3 | ±3.0% |
| 4 | ±2.5% |
| 5 | ±2.5% |
| 6 | ±2.5% |
| 7 | ±2.0% |
| 8 | ±2.0% |
| 1-8 | ±2.6% |

27. The tablet of claim 21, providing a Cₘₐₓ of about 1,300 nanograms per milliliter of serum.

28. The tablet of claim 26, wherein said Cₘₐₓ is between about 1,260 and about 1,325.

29. The tablet of claim 21, providing an AUC₀₋₂₄ of about 12,100 nanograms per milliliter per hour.

30. The tablet of claim 21, wherein said extended release tablet releases about 20-40% of the total amount of metformin hydrochloride by the end of the first hour; about 35-55% of the total amount of metformin hydrochloride by the end of the second hour; about 65-85% of the total amount of metformin hydrochloride by the end of the sixth hour; and at least about 85% of the total amount of metformin hydrochloride by the end of the tenth hour.

31. The tablet of claim 30, having a variability of drug release rate of not more than about ±3.5% during any one hour.

32. A pharmaceutical tablet comprising:
a. At least about one gram of metformin hydrochloride,
b. Hydrophilic material in an amount of not more than about 5% (w/w) of said metformin hydrochloride,
c. Said pharmaceutical tablet providing extended release, wherein the average hourly maintenance dose release rate is not more than about 15% of the total amount of metformin hydrochloride.

33. The tablet of claim 32, where the average hourly maintenance dose after the end of the second hour is not more than about 8% of the total amount of metformin hydrochloride per hour.

34. The tablet of claim 33, wherein the maintenance dose drug release rate generally decreases over time.

35. The tablet of claim 34, where the maintenance dose is approximately as follows:

| | Drug Release |
|---|---|
| Hour | (as % of Total) |
| 3 | 12.5 |
| 4 | 7.5 |
| 5 | 10.0 |
| 6 | 5.0 |
| 7 | 5.5 |
| 8 | 5.0 |
